(19) United States
(12) Patent Application Publication
(10) Pub. No.: US 2024/0287708 A1
(43) Pub. Date: Aug. 29, 2024

(54) GENERATING RECOMBINANT AFFINITY REAGENTS WITH ARRAYED TARGETS

(71) Applicants: ARIZONA BOARD OF REGENTS ON BEHALF OF ARIZONA STATE UNIVERSITY, Scottsdale, AZ (US); THE BOARD OF TRUSTEES OF THE UNIVERSITY OF ILLINOIS, Urbana, IL (US)

(72) Inventors: Joshua Labaer, Chandler, AZ (US); Kevin Gorman, Chicago, IL (US); Brian Kay, Chicago, IL (US); Jie Wang, Tempe, AZ (US); Ji Qiu, Chandler, AZ (US)

(21) Appl. No.: 18/417,216

(22) Filed: Jan. 19, 2024

Related U.S. Application Data
(63) Continuation of application No. 17/405,172, filed on Aug. 18, 2021, now Pat. No. 11,913,138.
Application No. 17/405,172 is a continuation of application No. 15/748,946, filed on Jan. 30, 2018, now Pat. No. 11,124,791.
(60) Provisional application No. 62/218,362, filed on Sep. 14, 2015.

(51) Int. Cl. C40B 30/04; C12N 15/10; C12Q 1/6811

(52) U.S. Cl. CPC C40B 30/04; C12N 15/1037; C12N 15/1041; C12N 15/1062; C12Q 1/6811

(57) ABSTRACT

Methods for screening of affinity reagents for many target proteins of interest simultaneously. Arrayed targets (e.g., peptide, protein, RNA, cell, etc.) are used in affinity selection experiments to reduce the amount of target needed and to improve the throughput of discovering recombinant affinity reagents to a large collection of targets.

### CROSS-REFERENCE TO RELATED APPLICATIONS

This application is a continuation of U.S. application Ser. No. 17/405,172, filed Aug. 18, 2021, which application is a continuation of U.S. application Ser. No. 15/748,946, filed Jan. 30, 2019, which application represents the U.S. National Stage entry of International Application No. PCT/US2016/051514, filed on Sep. 13, 2016, and claims priority to and the benefit of U.S. Provisional Application No. 62/218,362, filed on Sep. 14, 2015, the disclosures of which are incorporated herein by reference in their entireties.

### FIELD OF THE INVENTION

Embodiments herein relate generally to the field of high-throughput screening of affinity reagents for many targets of interest simultaneously.

### BACKGROUND OF THE INVENTION

Affinity selection is a process that utilizes so-called “display technologies” (ribosome-, mRNA-, and phage-display) to isolate recombinant affinity reagents for a given target (e.g., peptide, protein, RNA, cell, etc.). One of the bottlenecks of this process is producing the targets that are needed for the affinity selection.

In fact, many affinity reagent pipelines devote a significant amount of resources to generating high-quality target proteins. In addition, because the targets used in selection are sometimes labile (i.e., they are prone to degradation and denaturation), affinity selections fail.

Consequently, achieving a high-throughput and efficient affinity selection process remains problematic.

### SUMMARY OF THE INVENTION

Embodiments herein relate to cost-effective screening of affinity reagents for many target proteins of interest simultaneously. Consequently, affinity reagents can be discovered that will potentially aid in detecting, inhibiting, or activating target proteins.

In various embodiments, arrayed targets (e.g., peptide, protein, RNA, cell, etc.) are used in affinity selection experiments to reduce the amount of target needed and to improve the throughput of discovering recombinant affinity reagents to a large collection of targets.

Preferably, protein-target method embodiments herein use arrayed material that is translated shortly before each round of selection, as using labile protein targets is found to be less effective than using freshly made target samples.

These and other aspects of the embodiments disclosed herein will be apparent upon reference to the following disclosure.

### BRIEF DESCRIPTION OF THE DRAWINGS

FIG. 1. Affinity selection via phage-display using arrayed targets. First, the naïve library is incubated with freshly-translated target on the array. Next a wash step removes non-binding clones from the array, while binding clones are retained. Phage particles remaining on the array are then eluted from their respective targets and are amplified for subsequent rounds.

FIG. 2. Proposed pipeline for identifying recombinant affinity reagents.

FIG. 3. Detecting phage particles on NAPPA array. (A) Schematic of array layout containing 13 unique targets. (B) Detection of phage particles (1:1000) displaying a known MAP2K5 binder. (C) Detection of freshly-translated protein in wells via the halo epitope.

FIG. 4. Phage library affinity selection of the protein targets MAP2K5, CTBP1, SARA1A, and CDK2. Phage Input is normalized by counting colonies of TG1 cells infected by each library. Approximately, R1-lib: R2-Lib: Naïve Lib=1:1:10.

FIG. 5. Graphs of colony counts for the naive library, Round 1, and Round 2 of selection for the protein targets of the experiment in FIG. 4.

FIG. 6. RPS6KA3 is an antigen that is not selected in the initial enrichment experiment. Therefore, we would not expect increased signal after two rounds of enrichment. Consistently, this data shows RPS6KA3 was negatively selected.

### DETAILED DESCRIPTION OF THE INVENTION

Embodiments herein relate to arrayed targets that are used in affinity selection of display libraries. Traditionally, affinity selection procedures use individual protein or peptide as targets, which have a low throughput (i.e., one at a time) and require a significant amount of target.

With the advent of so-called “array” technologies, one is able to a) spot proteins or peptides on an array or b) synthesize in situ thousands of fresh target proteins on a solid surface (array) within a few hours. Thus, one solution to the low-throughput/large amount of target limitations is to affinity select with arrayed targets. For example, synthetic peptides or proteins can be spotted or captured in arrays on glass slides. Alternatively, proteins can be synthesized in situ in individual spots of an array. The method of choice of in vitro synthesis and capture of proteins in spots is the nucleic-acid programmable protein array (NAPPA).

In NAPPA, cDNAs coding for the target of interest are cloned into an expression vector, which generates a fusion (Halo Tag, GST, etc.) to the target, and spotted onto an aminosilane-coated glass slide. Then to each spot, a HeLa cell in vitro transcription-translation reagent is added, whereby the fusion gene is transcribed into mRNA and translated. The nascent proteins are captured to the slide with an antibody/affinity agent to the fusion partner (i.e., HaloTag-ligand, α-GST antibody) that is spotted adjacent to the DNA during the manufacture of the array. This method allows for up to thousands of protein targets to be arrayed.

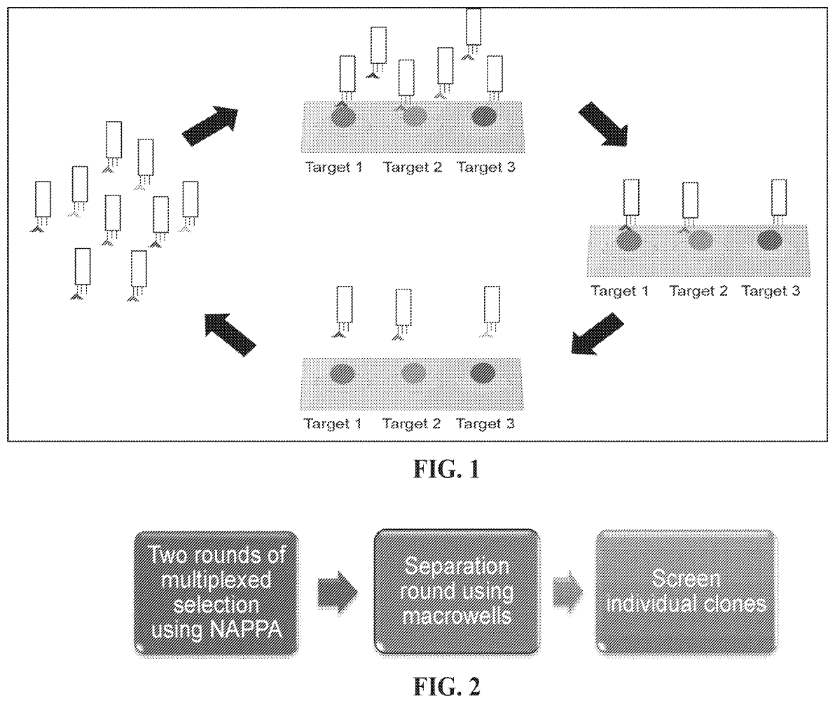

In the embodiments disclosed herein, targets, which have been generated by NAPPA, are used in affinity selection experiments (FIG. 1). By using freshly translated protein/peptide targets (i.e., within approximately 1 to 24 hours), the likelihood of denaturation of targets during storage and freeze thawing is reduced.

In addition, this process is performed on an array, which can produce many fresh protein samples. Therefore, one is able to perform a multiplexed selection on multiple targets using a single library, thereby reducing the time and cost of generating these reagents.

In some method embodiments, the process includes first performing two rounds of multiplexed panning on the array, followed by a separation round using a macrowell format (that still utilizes freshly-translated target protein), which separates binding phage based on their cognate target (FIG. 2). Finally, the resulting clones from pools isolated in the separation round are analyzed via macrowell analysis or ELISA to identify clones with the highest affinity for the target.

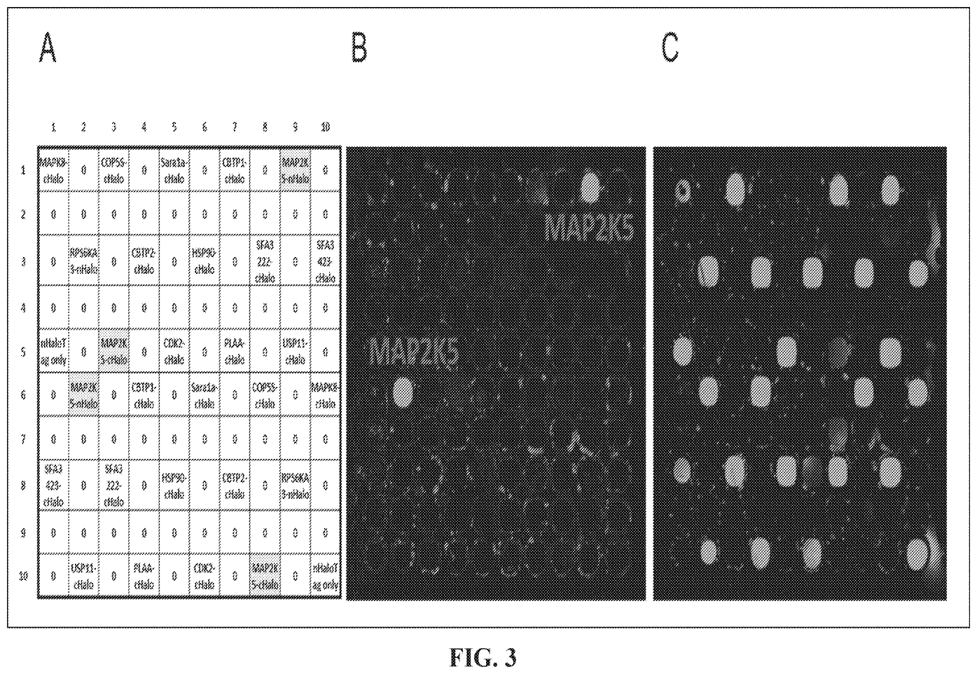

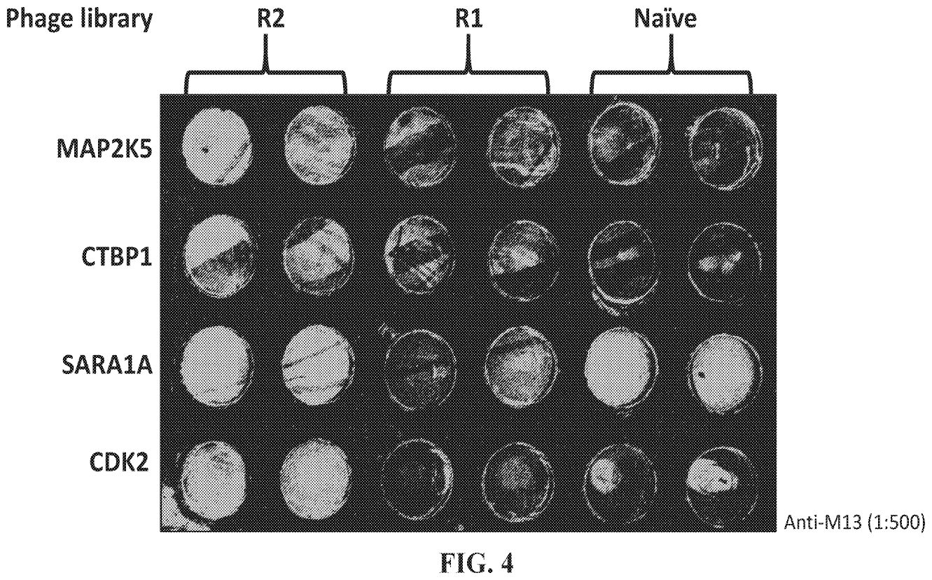

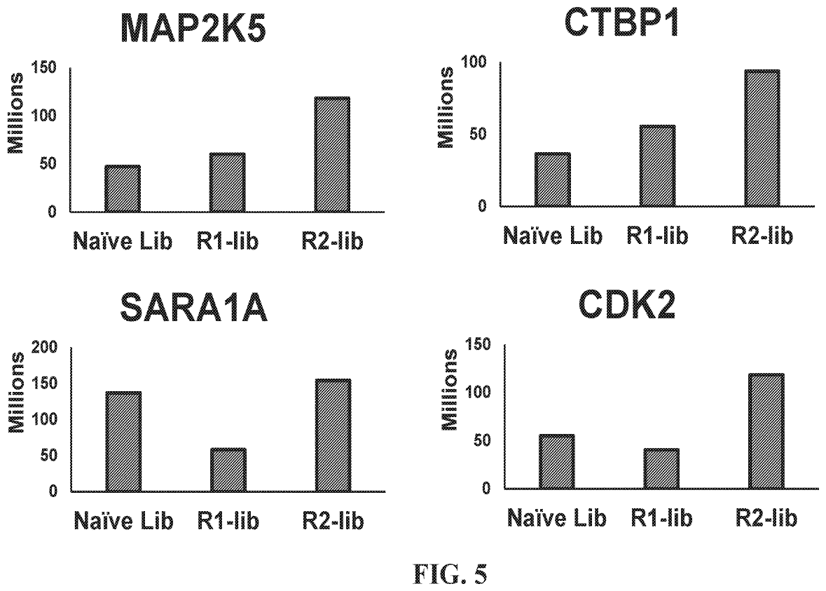

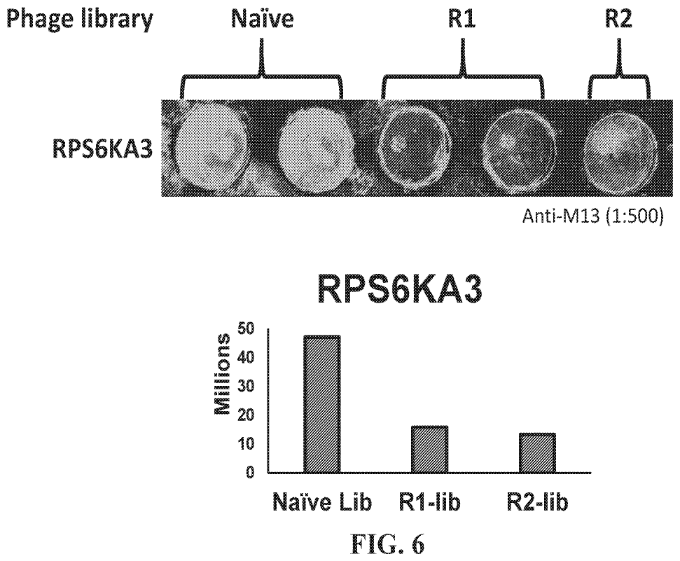

Recently, we have shown that an M13 bacteriophage, which displays a known binder to a particular protein target, can be detected to bind the NAPPA-generated and arrayed form of the same target (FIG. 3). A strong signal occurs when the virions are either pure or mixed 1 to 100 with a phage library containing 2×10¹⁰ members. This finding demonstrates that virions displaying a binder can bind selectively and efficiently to a NAPPA-generated target protein.

### Examples

### One Construction and Array Production

All genes of interest were cloned in pJFT7_nHALO or pJFT7_cHALO, the NAPPA compatible expression vectors. These expression vectors allow the in vitro expression of proteins of interest with a terminal HaloTag. Protein arrays were constructed through a contra capture concept as described (1).

### Enrichment

Array displaying MAP2K5, CTBP1, SARA1A and CDK2 were constructed and expressed. The initial non-enrichment phage library was incubated and washed to allow binding. Mild acid (0.2M Glycine pH2.0) wash was used to remove the bond phage particles and immediately neutralized using 1M Tris-Cl (pH9.1). E. coli were then infected with the collected phage for titring and amplification as previously described (2).

### Probe Libraries on Arrays

To evaluate the enrichment efficiency, same input of non-enriched library, R1 and R2 were probed on the protein microarray containing MAP2K5, CTBP1, SARA1A, CDK2 and RPS6KA3 for 1 hr at RT, followed by the M13 antibody at 1:500 dilution for another 1 hr at room temperature. Alexa Fluor 647 or Alexa Fluor 555 conjugated anti-mouse IgG secondary antibodies (Thermo Scientific) were then incubated with the array for 1 hr. After proper washing, slides were scanned at 10 micron resolution using TECAN scanner.

### REFERENCES

1. Karthikeyan K, Barker K, Tang Y, Kahn P, Wiktor P, Brunner A, Knabben V, Takulapalli B, Buckner J, Nepom G, LaBaer J, Qiu J. A Contra Capture Protein Array Platform for Studying Post-translationally Modified (PTM) Auto-antigenomes. Mol Cell Proteomics. 2016 July; 15(7):2324-37. doi: 10.1074/mcp.M115.057661. Epub 2016 May 2. PubMed PMID: 27141097; PubMed Central PMCID: PMC4937507.
2. Kay, B. K. et al., eds. Phage display of peptides and proteins: a laboratory manual

The following claims are not intended to be limited to the embodiments and other details provided herein. All references disclosed herein are hereby incorporated by reference in their entirety.

What is claimed is:

1. A method for affinity selection with arrayed target material, comprising the steps of:
a) incubating a display material with said arrayed target material,
b) washing said arrayed target material to removes non-binding display material from the arrayed target material,
c) eluting from the arrayed target material any bound display material,
d) amplifying said bound display material; and
e) performing at least a further round of affinity selection based on steps a)-c).

2. The method of claim 1, wherein said array target material comprises a peptide, protein, RNA, DNA or cell.

3. The method of claim 1, wherein said display material comprises a ribosome-, mRNA-, or phage-display.

4. The method of claim 1, further comprising the step of isolating binding display material and analyzing via macrowell analysis or ELISA to identify display material with the highest affinity for the array target material.

5. A method for affinity selection with arrayed target protein, comprising the steps of:
a) incubating a phage display material with said arrayed target protein,
b) washing said arrayed target protein to removes non-binding phage display material from the arrayed target material,
c) eluting from the arrayed target protein any bound phage display material,
d) amplifying said bound phage display material; and
e) performing at least a further round of affinity selection based on steps a)-c).

6. The method of claim 5, wherein said array target protein comprises freshly-translated protein.

7. The method of claim 5, wherein said display material comprises a ribosome-, mRNA-, or phage-display.

8. The method of claim 5, wherein said array target protein comprises one or more of MAP2K5, CTBP1, SARA1A, CDK2 and RPS6KA3.

9. The method of claim 5, further comprising the step of isolating binding display material and analyzing via macrowell analysis or ELISA to identify display material with the highest affinity for the array target material.

10. A method for negative selection of an arrayed target material, comprising the steps of:
a) incubating a display material with said arrayed target material,
b) washing said arrayed target material to removes non-binding display material from the arrayed target material,
c) eluting from the arrayed target material any bound display material,
d) amplifying said bound display material;
e) performing at least a further round of affinity selection based on steps a)-c); and
f) analyzing via macrowell analysis or ELISA to identify display material with decreasing affinity for the array target material versus a first round of selection.
